(19) United States
(12) Patent Application Publication
(10) Pub. No.: US 2005/0232901 A1
(43) Pub. Date: Oct. 20, 2005

(54) Materials and methods for treating or preventing oxalate-related disease

(76) Inventor: Ousama M. Zaghmout, Lorton, VA (US)

Correspondence Address: Dr. O. M. (Sam) Zaghmout, 8509 Kemon Ct, Lorton, VA, 22079, US

(21) Appl. No.: 11/105,493

(22) Filed: Apr. 14, 2005

Related U.S. Application Data
(60) Provisional application No. 60/561,897, filed on Apr. 14, 2004.

(51) Int. Cl. A61K 45/00; C12N 1/21

(52) U.S. Cl. 424/93.4

(57) ABSTRACT

A method for treating, preventing, controlling, or impeding a disease in a human and non human wherein said method comprises administering to said human a composition comprising a material selected from the group consisting of oxalate-degrading microbes and oxalate-degrading enzymes. A composition for treating, preventing, controlling, or impeding a disease in a human and non human wherein said composition comprises a material selected from the group consisting of oxalate-degrading microbes and oxalate-degrading enzymes formulated in a food product for human oral consumption.

### CROSS-REFERENCE TO A RELATED APPLICATION

This application claims the benefit of Provisional Patent Application Ser. No. 60/561,897 Filed Apr. 14, 2004.

### TECHNICAL FIELD

This invention relates to the approach of decomposing and removing oxalic acid.

This invention relates to a method for reducing or alleviating diseases that are associated with excess oxalate which include, but not limited to, osteoporosis, kidney-urinary tract stone disease (urolithiasis), vulvodynia, oxalosis associated with end-stage renal disease, cardiac conductance disorders, Crohn's disease, and other enteric disease states.

The present invention relates also to nutritional or pharmaceutical compositions comprising extracts or concentrates of certain plants and their use to treat bone disorder.

The invention relates to a composition for treating a disease comprising oxalate degrading enzyme and at least one of a carrier wherein said composition is adapted to be administered to human and non-human on a periodic basis in less than a lethal dosage.

This invention also relates to a method for reducing oxalate concentrations in a plant wherein said method comprises producing a transgenic plant that express polynucleotides encoding a oxalate-degrading enzymes.

This invention relates to a salad comprising a plant material capable of being ingested for its nutritional value, said plant wherein said salad is used for treating, preventing, controlling, or impeding a disease.

This invention also relates to a method for lowering the level of oxalic acid and/or the amount of precipitated oxalate in the environment, wherein said method comprises the use of a plant, plant cell or any plant part.

### BACKGROUND OF THE INVENTION

Oxalic acid (and/or its salt-oxalate) is a highly toxic natural by-product of catabolism in vertebrate animals and many consumable plants. Oxalic acid is strong dicarboxylic acid. Unfortunately, a significant portion of humans are unable to properly metabolizing oxalate, a condition which may result in the formation of kidney stones and bone disorders in those persons.

It is estimated that 70% of all kidney stones are composed of some amount of oxalate. Approximately 12 percent of the U.S. population will suffer from a kidney stone at some time in their lives, and the incidence is rising not only in the United States, but also in other countries such as Sweden and Japan (Curhan, 1993. “A Prospective study of dietary calcium and other nutrients and the risk of symptomatic kidney stones,” N. E. J. Med. 328:833-838). Moreover, although a healthy person breaks down or excretes sufficient quantities of oxalate to avoid excessive accumulation of oxalate in the tissues, a number of disease states are known to be associated with malfunctions of oxalate metabolism, These include, urolithiasis, vulvodynia, oxalosis associated with end-stage renal disease, pyridoxine deficiency, cardiac conductance disorders, Crohn's disease, and other enteric disease states. Urolithiasis is associated with stones that are composed of calcium oxalate alone or calcium oxalate plus calcium phosphate.

The risk for formation of kidney stones revolves around a number of factors that are not yet completely understood. Kidney-urinary tract stone disease occurs in as much as 12% of the population in Western countries and about 70% of these stones are composed of calcium oxalate or of calcium oxalate plus calcium phosphate. Some individuals (e.g., patients with intestinal disease such as Crohn's disease, inflammatory bowel disease, or steatorrhea and also patients that have undergone jejunoileal bypass surgery) absorb more of the oxalate in their diets than do others. For these individuals, the incidence of oxalate urolithiasis increases markedly. The increased disease incidence is due to increased levels of oxalate in kidneys and urine, and this, the most common hyperoxaluric syndrome in man, is known as enteric hyperoxaluria. Oxalate is also a problem in patients with end-stage renal disease and there is recent evidence (Solomons, C. C., M. H. Melmed, S. M. Heitler [1991] “Calcium citrate for vulvar vestibulitis” Journal of Reproductive Medicine 36:879-882) that elevated urinary oxalate is also involved in vulvar vestibulitis (vulvodynia).

Osteoporosis is another disease that is associated with excess oxalate. It is well established that a low calcium diet can lead to osteoporosis. Most calcium in the diet remains in the gut where it binds to oxalate from food and the liver. The bound oxalate cannot be absorbed and is excreted in the faeces. This means that only little amount of the calcium in the diet will be available for making bones. In that respect, osteoporosis can be prevented and treated by controlling the amount of oxalate thereby increasing the bioavailability of calcium.

Osteoporosis. is a disease characterized by low bone mass and structural deterioration of bone tissue, leading to bone fragility and an increased susceptibility to fractures of the hip, spine, and wrist. In that respect, osteoporosis weakens bones and can lead to painful and debilitating bone fractures. In humans, osteoporosis is a common feature of aging. Men as well as women suffer from osteoporosis, Loss of bone starts in women at the time of the menopause and in men at about age 55 and leads to an increase in fracture rates in both sexes. Ten million Americans have the disease and 18 million have low bone mass, placing them at risk, says the National Osteoporosis Foundation (NOF). The NOF says osteoporosis causes 1.5 million fractures a year and costs the U.S. health-care system about $14 billion annually. Osteoporosis-related vertebral fractures may lead to stooped posture, loss of height and chronic pain and disability. It also may cause compression of the lungs and stomach. Hip fractures can be life threatening, with a 20 percent increase in mortality within a year after suffering a hip fracture.

It is believed that oxalic acid or oxalate may be one of the primary causes of the onset of osteoporosis. Too much oxalic acid or oxalate in the diet may reduce the calcium in the body to a point of causing or aggravating osteoporosis. Hence, the treatment and prevention of osteoporosis is the control of the oxalic acid, oxalate and calcium levels. Women in a high risk group for osteoporosis should be careful to increase calcium intake and decrease oxalic acid or oxalate intake to prevent, treat, or control osteoporosis.

Oxalic acid is found in a wide diversity of foods, and is therefore, a component of many constituents in human and animal diets. Increased oxalate absorption may occur after foods containing elevated amounts of oxalic acid are eaten. Foods such as spinach and rhubarb are well known to contain high amounts of oxalate, but a multitude of other foods and beverages also contain oxalate. Because oxalate is found in such a wide variety of foods, diets that are low in oxalate and which are also palatable are hard to formulate. In addition, compliance with a low oxalate diet is often problematic.

The top 8 foods or plants known to increase urinary oxalate are identified by Massey et (Massey, L. K., H. Roman-Smith, and R. A. L. Sutton (1993) Effect of dietary oxalate and calcium on urinary oxalate and risk of formation of calcium oxalate kidney stones, Journal of the American Dietetic Association. 93: 901-906) as rhubarb, spinach, beets, nuts, chocolate, strawberries, wheat bran, and tea. Other foods or plants containing oxalate include leafy green vegetables, asparagus, runner beans, beetroot, brussel sprouts, cabbage, carrots, cauliflower, celery, chives, lettuce, marrow, mushrooms, onions, parsley, green peas, potatoes, radishes, rhubarb, spinach, tomatoes, turnips, apples, apricots, ripe bananas, gooseberries, grapefruits, melons, oranges, peaches, pears, pineapples, plums, blueberries, raspberries, strawberries, arugula, beet greens, collard greens, kale, endive, bok choy, dandelion greens, escarole, cole, mache, mustard greens, radicchio, rapini, swiss chard, and watercress.

There have been cases of oxalate-poisoning, especially from the species rhubarb (Rlieicrn rhaponticum, L.) and sorrel grass (Rllsnex acetosa L.). In high levels, oxalic acid can be corrosive to the gastrointestinal tract (Hodgkinson, (1977) Oxalic Acid in Biology and Medicine, Academic Press, London). Cattle have been poisoned by high oxalate content of Settzi-ia found in grazing areas. However, workers in Australia found a cultivar of Setaria cattle can graze safely (Hodgkinson, (1977) Oxalic Acid in Biology and Medicine, Academic Press, London.).

Endogenous oxalate is also produced metabolically by normal tissue enzymes. Oxalate (dietary oxalate that is absorbed as well as oxalate that is produced metabolically) is not further metabolized by tissue enzymes and must therefore be excreted. This excretion occurs mainly via the kidneys. The concentration of oxalate in kidney fluids is critical, with increased oxalate concentrations causing increased risk for the formation of calcium oxalate crystals and thus the subsequent formation of kidney stones.

Bacteria that degrade oxalate have also been isolated from human feces (Allison, M. J., H. M. Cook, D. B. Milne, S. Gallagher, R. V. Clayman [1986] “Oxalate degradation by gastrointestinal bacteria from humans” J. Nutr. 116:455-460). These bacteria were found to be similar to oxalate-degrading bacteria that had been isolated from the intestinal contents of a number of species of animals (Dawson, K. A., M. J. Allison, P. A. Hartman [1980] “Isolation and some characteristics of anaerobic oxalate-degrading bacteria the rumen” Appl. Environ. Microbiol. 40:833-839; Allison, M. J., H. M. Cook [1981] “Oxalate degradation by microbes of the large bowel of herbivores: the effect of dietary oxalate” Science 212:675-676; Daniel, S. L., P. A. Hartman, M. J. Allison [1987] “Microbial degradation of oxalate in the gastrointestinal tracts of rats” Appl. Environ. Microbiol. 53:1793-1797). These bacteria are different from any previously described organism and have been given both a new species and a new genus name (Allison, M. J., K. A. Dawson, W. R. Mayberry, J. G. Foss [1985] “Oxalabacter formigenes gen. nov., sp. nov.: oxalate-degrading anaerobes that inhabit the gastrointestinal tract” Arch. Microbiol. 141:1-7).

Not all humans carry populations of O. formigenes in their intestinal tracts (Allison, M. J., S. L. Daniel, N. A. Comick [1995] “Oxalate-degrading bacteria” In Khan, S. R. (ed.), Calcium Oxalate in Biological Systems CRC Press; Doane, L. T., M. Liebman, D. R. Caldwell [1989] “Microbial oxalate degradation: effects on oxalate and calcium balance in humans” Nutrition Research 9:957-964). There are low concentrations or a complete lack of oxalate degrading bacteria in the fecal samples of persons who have had jejunoileal bypass surgery (Allison et al. [1986] “Oxalate degradation by gastrointestinal bacteria from humans” J. Nutr. 116:455-460). Also, certain humans and animals may maintain colonies of O. formigenes but nevertheless have excess levels of oxalate for reasons which are not clearly understood.

Genes encoding enzymes which that degrade oxalate have also been isolated from a number of plant species including wheat. Transgenic plants that express oxalate oxidase have been established (e.g., Zaghmout et al. Abstract. Annual meeting of Plant Physiology. 1997, also see U.S. Pat. No. 6,380,461, U.S. Pat. No. 6,380,460, U.S. Pat. No. 6,376,748, U.S. Pat. No. 6,376,748). However, the use of a composition comprising a transgenic plant or plant extracts or plant parts that overexpress oxalate oxidase in reducing the incidence of a disease or alleviating the symptomes have never been explored. In particular preventing osteoporosis generating accompanying aging as well as above mentioned diseases.

The present invention relates also to nutritional or pharmaceutical compositions comprising extracts or concentrates of certain plants and their use to treat bone disorder.

The invention relates to a composition for treating a disease comprising oxalate degrading enzyme and at least one of a carrier wherein said composition is adapted to be administered to human and non-human on a periodic basis in less than a lethal dosage.

This invention also relates to a method for reducing oxalate concentrations in a plant wherein said method comprises producing a transgenic plant that express polynucleotides encoding a oxalate-degrading enzymes.

This invention relates to a salad comprising a plant material capable of being ingested for its nutritional value, said plant wherein said salad is used for treating, preventing, controlling, or impeding a disease.

This invention also relates to a method for lowering the level of oxalic acid and/or the amount of precipitated oxalate in the environment, wherein said method comprises the use of a plant, plant cell or any plant part.

### SUMMARY

This invention relates to the approach of decomposing and removing oxalic acid.

This invention relates to a method for reducing or alleviating diseases that are associated with excess oxalate which include, but not limited to, osteoporosis, kidney-urinary tract stone disease (urolithiasis), vulvodynia, oxalosis associated with end-stage renal disease, cardiac conductance disorders, Crohn's disease, and other enteric disease states.

The present invention relates also to nutritional or pharmaceutical compositions comprising extracts or concentrates of certain plants and their use to treat bone disorder.

The invention relates to a composition for treating a disease comprising oxalate degrading enzyme and at least one of a carrier wherein said composition is adapted to be administered to human and non-human on a periodic basis in less than a lethal dosage.

This invention also relates to a method for reducing oxalate concentrations in a plant wherein said method comprises producing a transgenic plant that express polynucleotides encoding a oxalate-degrading enzymes.

This invention relates to a salad comprising a plant material capable of being ingested for its nutritional value, said plant wherein said salad is used for treating, preventing, controlling, or impeding a disease.

This invention also relates to a method for lowering the level of oxalic acid and/or the amount of precipitated oxalate in the environment, wherein said method comprises the use of a plant, plant cell or any plant part.

### DETAILED DESCRIPTION

The subject invention pertains to the introduction of oxalate-degrading bacteria and/or an oxalate-degrading enzyme and/or pharmaceutical composition and/or nutriceutical composition and/or food composition and/or salad composition into a human or animal intestinal tract where the activity of the composition(s) reduces the amount and/or concentration of oxalate present thereby reducing the risk of disease due to oxalate. Also, this invention relates to a method and a composition for lowering the level of oxalic acid and/or the amount of precipitated oxalate in the environment, wherein said method or composition comprises the use of a plant, plant cell or any plant part.

In a specific embodiment, the subject invention pertains to the preparation and administration of cells of a plant that expresses or overexpresses an oxalate-degrading enzyme and/or oxalate-degrading bacteria of the species, Oxalobacter formigenes, to the human or animal intestinal tract where the activity oxalate present in the intestine thereby will be reduced which leads to an increase in the availability of calcium and also a reduction of concentrations of oxalate in the kidneys and in other cellular fluids. The introduced cells of oxalate-degrading bacteria degrade oxalate and replicate in the intestinal habitat so that progeny of the initial cells colonize the intestine and continue to remove oxalate. This activity of oxalate-degrading enzyme expressing or overexpressing plants and oxalate-degrading bacteria reduce the risk for formation of kidney stones as well as other disease complications caused by oxalic acid. In a preferred embodiment for human use, the specific strains of O. formigenes used are strains isolated from human intestinal samples. The strains are thus part of the normal human intestinal bacterial flora. However, since they are not present in all persons, or are present in insufficient numbers, the introduction of these organisms corrects a deficiency that exists in some humans.

Pharmaceutical and nutriceutical compositions for the introduction of oxalate degrading bacteria and/or enzymes into the intestine include bacteria and/or enzymes that have been lyophilized or frozen in liquid or paste form and encapsulated in a gel capsule or other enteric protection. The gel cap material is preferably a polymeric material which forms a delivery pill or capsule that is resistant to degradation by the gastric acidity and enzymes of the stomach but is degraded with concomitant release of oxalate-degrading materials by the higher pH and bile acid contents in the intestine. The released material then converts oxalate present in the intestine to harmless products. Pharmaceutical or nutriceutical carriers also can be combined with the bacteria or enzymes. These would include, for example, saline-phosphate buffer.

Bacteria and/or enzymes to be administered can be delivered as capsules or microcapsules designed to protect the material from adverse effects of acid stomach. One or more of several enteric protective coating methods can be used. Descriptions of such enteric coatings include the use of cellulose acetate phthalate (CAP) (Yacobi, A., E. H. Walega, 1988, Oral sustained release formulations: Dosing and evaluation, Pergammon Press). Other descriptions of encapsulation technology include U.S. Pat. No. 5,286,495, which is incorporated herein by reference. The compositions of the subject invention can also be formulated as suppositories.

Other methods of administration of these microorganisms and/or enzymes and/or plant extracts containing oxalate-degrading enzymes to the intestines include adding the material directly to food sources. The bacteria may be added as freshly harvested cells, freeze dried cells, or otherwise protected cells. Foods may be supplemented with oxalate degrading organisms without affecting their taste or appearance. These foods may be, for example, yogurt, milk, peanut butter or chocolate. Upon ingestion, when the food products are being digested and absorbed by the intestines, the microorganisms and/or enzymes or the plant extracts degrade oxalate present in the intestines thus reducing absorption of oxalate into the blood stream.

As noted above, a variety of foods can be supplemented with oxalate degrading microorganisms. As an initial step, the microbes can be grown in media and separated from the media by, for example, centrifugation. Traditional yogurt cultures obtained from a commercial dairy can be mixed with the oxalate degrading microbial culture. This mixture of cultures then can be added to the basic dairy yogurt premix without adversely affecting taste or consistency. The yogurt can then be produced and packaged using traditional commercial procedures. In another example, the oxalate degrading bacteria can be added to already produced yogurts.

Another example is to add the microbes or the enzymes to milk after it has been homogenized and sterilized. Such a method is currently used in the diary industry for adding Lactobacillus acidophilis organisms to milk. Any food source containing bacteria can be used by supplementing with oxalate-degrading bacteria. These food products include cheese or meat products that have desirable microorganisms added during processing.

In yet a further embodiment, the subject invention provides a novel enzyme delivery system. This system comprises a plant which has been transformed with heterologous polynucleotide(s) to express oxalate-degrading enzymes. The enzyme-expressing transgenic plant may be administered to patients as a constituent of a salad, for example. Further, the enzyme-expressing plant may be administered to animals as a constituent of feed, for example, or grown in grazing pasture. The animals to which these products may be fed include, for example, cattle, pigs, dogs and cats.

Thus, as an alternative method of administration to the intestine, plants are genetically engineered to express oxalate-degrading enzymes. These transgenic plants are added to the diet, with the activity of the enzymes causing a decrease in the presence of oxalate. DNA sequences encoding these enzymes are known to those skilled in the art and are described in, for example, Zaghmout et al (Plant Physiology annual meeting. 1987. Abstract Number:1152), U.S. Pat. No. 6,441,275, U.S. Pat. No. 6,403,861, U.S. Pat. No. 6,380,461, U.S. Pat. No. 6,380,460, U.S. Pat. No. 6,376,748 and WO 98/16632.

In addition to plants which can be used as a dietary component to promote healthy oxalate levels in humans or animals, the subject invention provides plants with enhanced resistance to microbial infections (e.g., Sclerotinia sclerotorium). Specifically, the transformed plants of the subject invention are protected against microbes which require or use the presence of oxalate for plant pathogenicity. The plants of the subject invention, which are transformed to express oxalate-degrading enzymes are protected against, for example, certain fungi which need oxalate for pathogenicity. The genes encoding the enzymes can be modified to enhance expression and/or stability in plants. Also, the expression may be under the control of promoters which direct expression in particular tissues. Transgenic plants produced in this invention are tolerant or resistant to oxalate and non-oxalate producing fungi and other microorganisms such as bacteria, virus and others. More details about transgenic plants that express or overexpress genes encoding oxalate degrading enzymes can be found in U.S. Pat. No. 6,441,275, U.S. Pat. No. 6,403,861, U.S. Pat. No. 6,380,461, U.S. Pat. No. 6,380,460, U.S. Pat. No. 6.376.748 and WO 98/16632. The full contents of these patents are incorporated therein by a reference.

In one embodiment, the strains of bacteria (O. formigenes) used according to the subject invention are pure cultures that are isolated from anaerobic cultures that have been inoculated with dilutions of intestinal contents from normal humans or, for use with animals, from normal animals. A special calcium oxalate containing medium that allows detection of oxalate degrading colonies can be used. In one embodiment, the purity of each strain can be assured through the use of at least two subsequent repetitive cloning steps.

Strains of O. formigenes useful according to the subject invention have been characterized based upon several tests, these include: patterns of cellular fatty acids, patterns of cellular proteins, DNA and RNA (Jensen, N. S., M. J. Allison (1995) “Studies on the diversity among anaerobic oxalate degrading bacteria now in the species Oxalobacter formigenes” Abstr. to the General Meeting of the Amer. Soc. Microbiol., 1-29), and responses to oligonucleotide probes (Sidhu et al. 1996. “Detection and Characterization of Oxalobacter formigenes Strains Using Oligonucleotide Probes” Meeting for Urolithasis, pp 537-539, Pak, C.Y.C. et al. (ed). Two groups of these bacteria (Groups I and II, both existing within the description in U.S. Pat. No. 6,699,469) have been described. Strains used have been selected based upon oxalate degrading capacity, and evidence of the ability to colonize the human intestinal tract. Strains selected include representatives of both Groups I and II of the species as detailed in U.S. Pat. No. 6,699,469.

One embodiment of the present invention involves procedures for selection, preparation and administration of the appropriate oxalate-degrading bacteria to a diversity of subjects. Prominently, but not exclusively, these are persons or animals which do not harbor these bacteria in their intestines. These non-colonized or weakly-colonized persons or animals are identified using tests that allow for rapid and definitive detecting of O. formigenes even when the organisms are at relatively low concentrations in mixed bacterial populations such as are found in intestinal contents. The methods of the subject invention can also be used to treat individuals or animals whose oxalate-degrading bacteria have been depleted due to, for example, antibiotic treatment or in post-operative situations. The methods of the subject invention can also be used to treat individuals or animals who have colonies of oxalate-degrading bacteria but who still have unhealthy levels of oxalate due to, for example, oxalate susceptibility and/or excessive production of endogenous oxalate.

Bacteria which can be used according to the subject invention can be identified by at least two methods:
1) Oligonucleotide probes specific for these bacteria can be used; and/or
2) A culture test wherein an anaerobic medium with 10 mM oxalate is inoculated and after incubation at 37[deg.] C. for 1 to 7 days, the loss of oxalate is determined.

Pure cultures of O. formigenes strains can be grown in large fermenter batch cultures and cells can be harvested using techniques known to those skilled in the art. Cells from a selected single strain or mixtures of known strains can be treated as needed (e.g., freeze dried with trehalose or glycerol) to preserve viability and are then placed in capsules designed to protect the cells through their passage through the acid stomach (enteric coated capsules).

Cells are ingested in quantities and at intervals determined by the needs of individuals. In some cases a single, or periodic, use may be all that is needed and in other cases regular ingestion (e.g., with meals) may be needed.

The invention further pertains to administration to the human or animal intestinal tract of oxalate-degrading products or enzymes prepared from O. formigenes cells. In one embodiment, oxalate degrading enzymes can be purified and prepared as a pharmaceutical or nutriceutical or a food or a salad composition for oral consumption. In a preferred embodiment, these enzymes are produced recombinantly. DNA sequences encoding these enzymes are known to those skilled in the art and are described in, for example, WO 98/16632. These sequences, or other sequences encoding oxalate-degrading proteins, can be expressed in a suitable host. The host may be, for example, E. coli or Lactobacillus. The transformed host would included appropriate regulatory and transporter signals. The expressed protein may be isolated, purified and administered as described herein. Alternatively, the recombinant host expressing the desired oxalate-degrading proteins may be administered. The recombinant host may be administered in either a viable or non-viable form. In another preferred embodiment, the enzymes are coated or otherwise formulated or modified to protect the enzymes so that they are not inactivated in the stomach, and are available to exert their oxalate-degrading activity in the small intestine. Examples of such formulations are known to those skilled in the art and are described in, for example, U.S. Pat. No. 5,286,495.

The use of O. formigenes is particularly advantageous because it is an anaerobe that does not grow in aerobic tissue environments and does not produce any compounds which are toxic to humans or animals. As an alternative to either O. formigenes or a recombinant host, other oxalate-degrading bacteria may be used, such as Clostridium or Pseudomonas. Oxalate-degrading enzymes prepared from such alternative bacteria may be administered or the entire microbe may be administered.

In addition, all aforementioned embodiments are applicable to domesticated, agricultural, or zoo-maintained animals suffering from deficient numbers of oxalate-degrading bacteria, as well as to humans. For example, oxalate-degrading enzymes and/or microbes may be administered to house pets such as dogs, cats, rabbits, ferrets, guinea pigs, hamsters and gerbils, as well as to agricultural animals, such as horses, sheep, cows, poultry (e., chickens) and pigs.

In one aspect of the invention, the claims are:

1. A method for treating, preventing, controlling, or impeding a disease in a human and non human wherein said method comprises administering to said human a composition comprising a material selected from the group consisting of oxalate-degrading microbes and oxalate-degrading enzymes.

2. The method, according to claim 1, wherein said method comprises administration of oxalate-degrading enzymes, wherein the oxalate degrading enzyme is selected from the group consisting of formyl-CoA transferase, oxalyl-CoA decarboxylase, oxalic acid oxidase and oxalate decarboxylase.

3. The method, according to claim 1, wherein said oxalate-degrading enzymes are obtained from bacteria, fungi or plant.

4. The method, according to claim 3, wherein said oxalate-degrading enzymes are derived from bacteria of the group consisting of Clostridium, Pseudomonas, and oxalobacter.

5. The method, according to claim 2, wherein said enzymes are produced recombinantly.

6. The method, according to claim 5, wherein said enzymes are produced recombinantly in Escherichia coli or Agroabcterium spp.

7. The method, according to claim 2, which comprises administering formyl-CoA transferase and oxalyl-CoA decarboxylase, oxalic acid oxidase and/or oxalate decarboxylase.

8. The method, according to claim 7, wherein said enzymes are produced recombinantly.

9. The method, according to claim 2, wherein said oxalate-degrading enzymes are expressed in plants which have been transformed with polynucleotides encoding said oxalate-degrading enzymes.

10. The method, according to claim 1, wherein said method comprises administration of oxalate-degrading microbes.

11. The method, according to claim 10, wherein said oxalate-degrading microbes have been transformed with polynucleotides which encode said oxalate-degrading enzymes.

12. The method, according to claim 2, which further comprises administering an additional factor selected from the group consisting of oxalyl CoA, MgCl₂ and TPP.

13. The method, according to claim 10, which comprises administering whole viable oxalate-degrading microbes.

14. The method, according to claim 13, wherein said microbes are Oxalobacter formigenes.

15. The method, according to claim 13, wherein said microbes are selected from the group consisting of Clostridium and Pseudomonas.

16. The method, according to claim 13, wherein said microbes colonize the intestines.

17. The method, according to claim 1, which is used to treat a patient whose intestines have insufficient numbers of oxalate-degrading bacteria.

18. The method, according to claim 17, which is used to treat a patient whose natural intestinal bacteria have been depleted due to treatment with antibiotics.

19. The method, according to claim 1, which said microbe or said enzyme is formulated to reduce inactivation in the stomach.

20. The method, according to claim 19, wherein said formulation comprises a coating which dissolves preferentially in the small intestine compared to the stomach.

21. A composition for treating, preventing, controlling, or impeding
a disease in a human and non human wherein said composition comprises a material selected from the group consisting of oxalate-degrading microbes and oxalate-degrading enzymes formulated in a food product for human oral consumption.

22. The composition, according to claim 21, wherein said composition comprises whole, viable oxalate-degrading bacteria.

23. The method, according to claim 21, wherein said composition comprises cell lysate of oxalate-degrading bacteria.

24. The composition, according to claim 21, wherein said bacteria are Oxalobacter formigenes.

25. The composition, according to claim 21, wherein said bacteria are selected from the group consisting of Clostridium and Pseudomonas.

26. The composition, according to claim 21, wherein said composition comprises oxalate-degrading enzymes.

27. The composition, according to claim 26, wherein said enzymes are formyl-CoA transferase and oxalyl CoA decarboxylase, oxalic acid oxidase or oxalate decarboxylase.

28. The composition, according to claim 27, which further comprises a compound selected from the group consisting of oxalyl CoA, MgCl₂, and TPP.

29. The composition, according to claim 21, wherein said composition is formulated to reduce deactivation in the stomach.

30. The composition, according to claim 29, wherein said composition is coated with a material which preferentially degrades in the small intestine.

31. The method, according to claim 1, wherein said method is used to treat osteoporosis.

32. The method, according to claim 1, wherein said method is used to treat osteoporosis.

33. The method, according to claim 1, wherein the oxalate concentration is reduced in the intestines, blood, or urine.

34. A method for treating, preventing, controlling, or impeding
a disease in a human and non human wherein said method comprises administering to said human a composition comprising a material selected from the group consisting of oxalate-degrading microbes and oxalate-degrading enzymes; wherein the disease is selected from the group consisting of osteoporosis, urolithiasis, vulvodynia, and oxalosis and alike diseases.

35. The method, according to claim 34, wherein the oxalate-degrading enzyme is selected from the group consisting of a bacterium, a fungus, or a plant.

36. The method, according to claim 34, wherein said method comprises administration of oxalate-degrading enzymes wherein the oxalate de-grading enzymes is selected from the group consisting of formyl-CoA transferase, oxalyl-CoA decarboxylase, oxalic acid oxidase and oxalate decarboxylase.

In one of the embodiment of this invention, this invention relates to a food comprising transgenic plant material capable of being ingested for its nutritional value, said transgenic plant expressing an oxalate-degrading enzyme wherein said food is used for treating, preventing, controlling, or impeding a disease. In one aspect of the invention, The food can also be made from non-transgenic plant materials.

This invention also relates to a food comprising transgenic plant material capable of being ingested for its nutritional value, said transgenic plant expressing an oxalate-degrading enzyme wherein said food is used for treating, preventing, controlling, or impeding a disease, wherein the disease in human and non human.

This invention also relates to a food comprising transgenic plant material capable of being ingested for its nutritional value, said transgenic plant expressing an oxalate-degrading enzyme wherein said food is used to alleviate for treating, preventing, controlling, or impeding a disease, wherein the disease in human.

This invention also relates to a food comprising transgenic plant material capable of being ingested for its nutritional value, said transgenic plant expressing an oxalate-degrading enzyme wherein said food is used for treating, preventing, controlling, or impeding a disease, wherein the disease in animal.

This invention also relates to a food comprising transgenic plant material capable of being ingested for its nutritional value, said transgenic plant expressing an oxalate-degrading enzyme wherein said food is used for treating, preventing, controlling, or impeding a disease, wherein the enzyme of plant, bacterial or fungal origin.

This invention also relates to a food comprising transgenic plant material capable of being ingested for its nutritional value, said transgenic plant expressing an oxalate-degrading enzyme wherein said food is used to alleviate or reduce a disease, wherein the enzyme from a plant is oxalic acid oxidase.

This invention also relates to a food comprising transgenic plant material capable of being ingested for its nutritional value, said transgenic plant expressing an oxalate-degrading enzyme wherein said food is used for treating, preventing, controlling, or impeding a disease, wherein the enzyme from fungus is oxalate decarboxylase.

This invention also relates to a food comprising transgenic plant material capable of being ingested for its nutritional value, said transgenic plant expressing an oxalate-degrading enzyme wherein said food is used to alleviate or reduce a disease, wherein the enzyme from bacteria are formyl-CoA transferase and oxalyl-CoA decarboxylase.

This invention also relates to a food comprising transgenic plant material capable of being ingested for its nutritional value, said transgenic plant expressing an oxalate-degrading enzyme wherein said food is for treating, preventing, controlling, or impeding a disease, wherein the disease is osteoporosis, urolithiasis, vulvodynia, oxalosis associated with end-stage renal disease, cardiac conductance disorders, Crohn's disease, and other enteric disease states.

This invention also relates to a food comprising transgenic plant material capable of being ingested for its nutritional value, said transgenic plant expressing an oxalate-degrading enzyme wherein said food is used for treating, preventing, controlling, or impeding a disease, wherein the disease is osteoporosis.

This invention also relates to a food comprising transgenic plant material capable of being ingested for its nutritional value, said transgenic plant expressing an oxalate-degrading enzyme wherein said food is used for treating, preventing, controlling, or impeding a disease, wherein the disease is urolithiasis.

This invention also relates to a food comprising transgenic plant material capable of being ingested for its nutritional value, said transgenic plant expressing an oxalate-degrading enzyme wherein said food is used for treating, preventing, controlling, or impeding a disease, wherein the disease is vulvodynia.

This invention also relates to a food comprising transgenic plant material capable of being ingested for its nutritional value, said transgenic plant expressing an oxalate-degrading enzyme wherein said food is used for treating, preventing, controlling, or impeding a disease, wherein the disease is vulvodynia.

This invention also relates to a food comprising transgenic plant material capable of being ingested for its nutritional value, said transgenic plant expressing an oxalate-degrading enzyme wherein said food is used for treating, preventing, controlling, or impeding a disease, wherein the disease is oxalosis.

This invention also relates to a food comprising transgenic plant material capable of being ingested for its nutritional value, said transgenic plant expressing an oxalate-degrading enzyme wherein said food is used for treating, preventing, controlling, or impeding a disease, wherein the food is a salad, a fruit, a vegetable, a nut, jelly, a jam, a drink, and alike.

This invention also relates to a food comprising transgenic plant material capable of being ingested for its nutritional value, said transgenic plant expressing an oxalate-degrading enzyme wherein said food is used for treating, preventing, controlling, or impeding a disease, wherein the food is a soft drink or milk or milk products.

This invention also relates to a food comprising transgenic plant material capable of being ingested for its nutritional value, said transgenic plant expressing an oxalate-degrading enzyme wherein said food is used for treating, preventing, controlling, or impeding a disease, wherein fruit is an apple, blackberry, blueberry, chestnuts, chinkapin, fig, grapes, loquat, mayhaw, mulberry, nectarine, peach. pear, persimmon, plums pomegranate, quince, raspberry, citrus trees, mango, guavas, cherry, avocado, banana, carambola, cherimoya, currants, feijoa, Fig, gooseberry, jaboticaba, jackfruit, jujube, lychee, malabar, chestnut, mango, papaya, pineapple, raisin, sapodilla, tamarind, guava, white sapote, acerola, babaco, gooseberry, kiwifruit, loquat, macadamia, olive, passion fruit, pawpaw, pepino dulce, persimmon, pistachio, pomegranate, tamarillo.

This invention also relates to a food comprising transgenic plant material capable of being ingested for its nutritional value, said transgenic plant expressing an oxalate-degrading enzyme wherein said food is used for treating, preventing, controlling, or impeding a disease, wherein vegetable is rhubarb, spinach and beet, carrot, parsley, chives, beets and beet leaves, garlic, collards and radishes, tomato, potato, pepper, beans, okra, lettuce, chives, onion, garlic, and other vegetables containing oxalic acid or oxalate. Crop species such as, but not limited to, soybean also is part of the invention.

This invention also relates to a food comprising transgenic plant material capable of being ingested for its nutritional value, said transgenic plant expressing an oxalate-degrading enzyme wherein said food is used for treating, preventing, controlling, or impeding a disease, wherein nut is an almond, cashew, peanut, pecan, walnut, and nuts containing oxalic acid or oxalate.

This invention also relates to a food comprising transgenic plant material capable of being ingested for its nutritional value, said transgenic plant expressing an oxalate-degrading enzyme wherein said food is a cereal, a non-cereal plant or a grass. Also included in this invention plants such as coffee, cocoa, and tea.

This invention also relates to a food comprising transgenic plant material capable of being ingested for its nutritional value, said transgenic plant expressing an oxalate-degrading enzyme wherein said food is selected from the group consisting of: edible fruit, leaves, juices, roots, and seed of said plant.

This invention also relates to a food, wherein the food comprises beverages, infused foods, sauces, condiments, salad dressings, fruit juices, syrups, desserts, icings and fillings, soft frozen products, confections or intermediate food.

This invention also relates to a food, wherein the food further comprises an edible composition that is substantially a liquid.

This invention also relates to a food, wherein the food further comprises an edible composition that is substantially a solid.

This invention also relates to a food, wherein the food further comprises an edible composition that is a food supplement.

This invention also relates to a food, wherein the food further comprises an edible composition that is a nutraceutical.

This invention also relates to a food, wherein the food is administered to a human.

This invention also relates to a food, wherein the food is administered to a human. an animal.

This invention also relates to a food, wherein the food is administered to a human. livestock or poultry.

Also part of this invention is the use with the food a therapeutically effective dosage of a composition including at least one therapeutically effective compound for reducing the intake of oxalic acid or oxalate blockers such as citric acid, ascorbic acid, (vitamin C), pyridoxine hydrochloride (vitamin B6), calcium, alcohol, resins, clays, foods containing calcium, beverages containing alcohol, citric acid, or ascorbic acid, red meat or white meat of fowl containing pyridoxine hydrochloride, or other foods nutritional supplements or beverages containing oxalic acid or oxalate blockers.

Also part of this invention is a method for treating, preventing, controlling, or impeding a disease, comprising a food according to one of the claims of this invention.

Also part of this invention is a method comprising crossing a transgenic plant containing an exogenous gene which expresses an oxalate degrading enzyme with a plant which does not express an oxalate degrading enzyme, or at a plant which does not express sufficient amount of oxalate degrading enzyme.

This invention relates to a salad comprising a plant material capable of being ingested for its nutritional value, said plant wherein said salad is used for treating, preventing, controlling, or impeding a disease.

This invention also relates to a salad comprising a plant material capable of being ingested for its nutritional value, said transgenic plant expressing an oxalate-degrading enzyme wherein said salad is used for treating, preventing, controlling, or impeding a disease.

This invention also relates to a salad comprising a plant material capable of being ingested for its nutritional value, said transgenic plant expressing an oxalate-degrading enzyme wherein said salad is used for treating, preventing, controlling, or impeding a disease, wherein the disease in human and non human.

This invention also relates to a salad comprising a plant material capable of being ingested for its nutritional value, said plant expressing an oxalate-degrading enzyme wherein said salad is used for treating, preventing, controlling, or impeding a disease, wherein the disease in human, wherein the plant is a cereal plant.

This invention also relates to a salad comprising a plant material capable of being ingested for its nutritional value, said plant expressing an oxalate-degrading enzyme wherein said salad is used for treating, preventing, controlling, or impeding a disease, wherein the disease in human, wherein the cereal plant is selected from the group consisting of wheat, barley, oat, maize, sorghum, rice and any grass cultivated for its edible grain.

This invention also relates to a salad comprising a plant material capable of being ingested for its nutritional value, said transgenic plant expressing an oxalate-degrading enzyme wherein said food is used for treating, preventing, controlling, or impeding a disease, wherein the salad comprises additional, fruit, vegetable, meat, fish, chicken, milk products such as, but not limited to, cheese.

This invention also relates to a salad comprising a transgenic plant material capable of being ingested for its nutritional value, said transgenic plant expressing an oxalate-degrading enzyme wherein said salad is used for treating, preventing, controlling, or impeding a disease, wherein the plant is a transgenic plant, wherein the enzyme of plant, bacterial or fungal origin.

This invention also relates to a salad comprising a transgenic plant material capable of being ingested for its nutritional value, said transgenic plant expressing an oxalate-degrading enzyme wherein said salad is used for treating, preventing, controlling, or impeding a disease, wherein the enzyme from a plant is oxalic acid oxidase.

This invention also relates to a salad comprising a transgenic plant material capable of being ingested for its nutritional value, said transgenic plant expressing an oxalate-degrading enzyme wherein said salad is used for treating, preventing, controlling, or impeding a disease, wherein the enzyme from fungus is oxalate decarboxylase.

This invention also relates to a salad comprising a transgenic plant material capable of being ingested for its nutritional value, said transgenic plant expressing an oxalate-degrading enzyme wherein said salad is used for treating, preventing, controlling, or impeding a disease, wherein the enzyme from bacteria are formyl-CoA transferase and oxalyl-CoA decarboxylase.

This invention also relates to a salad comprising a plant material capable of being ingested for its nutritional value, said plant expressing an oxalate-degrading enzyme wherein said salad is used for treating, preventing, controlling, or impeding a disease, wherein the disease is osteoporosis, urolithiasis, vulvodynia, oxalosis associated with end-stage renal disease, cardiac conductance disorders, Crohn's disease, and other enteric disease states.

This invention also relates to a salad comprising a plant material capable of being ingested for its nutritional value, said transgenic plant expressing an oxalate-degrading enzyme wherein said food is used for treating, preventing, controlling, or impeding a disease, wherein the disease is osteoporosis.

This invention also relates to a salad comprising a transgenic plant material capable of being ingested for its nutritional value, said plant expressing an oxalate-degrading enzyme wherein said salad is used for treating, preventing, controlling, or impeding a disease, wherein the disease is urolithiasis.

This invention also relates to a salad comprising a plant material capable of being ingested for its nutritional value, said plant expressing an oxalate-degrading enzyme wherein said salad is used to for treating, preventing, controlling, or impeding a disease, wherein the disease is vulvodynia.

This invention also relates to a salad comprising a plant material capable of being ingested for its nutritional value, said plant expressing an oxalate-degrading enzyme wherein said salad is used for treating, preventing, controlling, or impeding a disease, wherein the disease is oxalosis.

This invention also relates to a salad comprising a plant material capable of being ingested for its nutritional value, said plant expressing an oxalate-degrading enzyme wherein said salad is used for treating, preventing, controlling, or impeding a disease, wherein the salad comprises an additional material that is selected from the group consisting of fruit, vegetable, nut, jelly, jam, drink, and others.

This invention also relates to a salad comprising plant material capable of being ingested for its nutritional value, said transgenic plant expressing an oxalate-degrading enzyme wherein said salad is used for treating, preventing, controlling, or impeding a disease, wherein the salad contains is a soft drink or milk or milk products.

This invention also relates to a salad comprising plant material capable of being ingested for its nutritional value, said plant expressing an oxalate-degrading enzyme wherein said salad is used for treating, preventing, controlling, or impeding a disease, wherein the food is a fruit.

This invention also relates to a salad comprising transgenic plant material capable of being ingested for its nutritional value, said plant expressing an oxalate-degrading enzyme wherein said salad is used for treating, preventing, controlling, or impeding a disease, wherein the said salad is a vegetable.

This invention also relates to a salad comprising plant material capable of being ingested for its nutritional value, said transgenic plant expressing an oxalate-degrading enzyme wherein said salad is for treating, preventing, controlling, or impeding a disease, wherein the salad further contains nut.

This invention also relates to a salad comprising plant material capable of being ingested for its nutritional value, said plant expressing an oxalate-degrading enzyme wherein the salad further comprises a non-cereal plant.

This invention also relates to a salad comprising transgenic plant material capable of being ingested for its nutritional value, said plant expressing an oxalate-degrading enzyme wherein said salad is selected from the group consisting of: edible fruit, leaves, juices, roots, and seed of said plant.

Also part of this invention is the use with the salad a therapeutically effective dosage of a composition including at least one therapeutically effective compound for reducing the intake of oxalic acid or oxalate blockers such as citric acid, ascorbic acid, (vitamin C), pyridoxine hydrochloride (vitamin B6), calcium, alcohol, resins, clays, foods containing calcium, beverages containing alcohol, citric acid, or ascorbic acid, red meat or white meat of fowl containing pyridoxine hydrochloride, or other foods nutritional supplements or beverages containing oxalic acid or oxalate blockers.

This invention also relates to a method for reducing oxalate concentrations in a plant wherein said method comprises producing a transgenic plant that express polynucleotides encoding a oxalate-degrading enzymes.

This invention also relates to a method, wherein said oxalate-degrading enzyme is of plant or fungal origin.

This invention also relates to a method, wherein said oxalate-degrading enzyme from plant origin is oxalic acid oxidase.

This invention also relates to a method, wherein said oxalate-degrading enzyme from fungus origin is oxalate decarboxylase.

This invention also relates to a plant produced by any of these methods.

This invention also relates to a plant tissue derived from a plant produced by any of these methods.

This invention also relates to a seed derived from a plant produced by any of these methods. Plants or plants parts resulting from said seeds.

This invention also relates to an embryo cell from the transgenic plant produced according any of the claims of this invention.

This invention also relates to a callus cell from the transgenic plant produced according any of the claims of this invention.

This invention also relates to a protoplast from the transgenic plant produced according any of the claims of this invention.

This invention also relates to a fruit from the transgenic plant produced according any of the claims of this invention.

This invention also relates to a vegetable from the transgenic plant produced according any of the claims of this invention.

This invention also relates to a leaf from the transgenic plant produced according any of the claims of this invention.

This invention also relates to a meristem from the transgenic plant produced according any of the claims of this invention.

This invention also relates to a plant resulting from a cross with any plant that is part of this invention. In addition, seeds resulting from these plants are also part of this invention.

This invention also relates to a method of reducing oxalate in a plant, the method comprising: a. transforming a plant or plant tissue with an oxalate degrading enzyme, wherein said plant tissue is characterized by having reduced oxalate than plants not-transformed with the oxalate-degrading enzyme; and b. expressing the oxalate degrading gene thereby making the plant contains reduced oxalate. In one aspect of the invention, these plants can be used treating, preventing, controlling, or impeding a disease. In one aspect of this invention, the disease is osteoporosis, urolithiasis, vulvodynia, oxalosis associated with end-stage renal disease, cardiac conductance disorders, Crohn's disease, and other enteric disease states. In one aspect of the invention, the oxalate degrading enzyme can be any of the enzymes mentioned in this invention and the alike enzymes which are can potentially degrade or mediate the degradation of oxalate. In one aspect of this invention, the plant can be any plant species. More preferably, the plant can a high, medium or low oxalate containing a plant. More preferably, the plant can be selected from the group consisting of spinach, carrot, parsley, rhubarb and other plant species. In one aspect of the invention, the oxalate-degrading enzyme can be from a gene that is isolated from a plant, bacteria or fungi. More preferably, the gene isolated from a plant encodes oxalic acid oxidase or the alike. More preferably, the gene isolated from fungus encodes oxalate decarboxylase. More preferably, the gene isolated from bacteria is formyl-CoA transferase and/or oxalyl-CoA decarboxylase.

In another embodiment, the invention relates to a composition for treating a disease comprising oxalate degrading enzyme and at least one of a carrier wherein said composition is adapted to be administered to human and non-human on a periodic basis in less than a lethal dosage.

The invention relates to a composition for treating a disease comprising oxalate degrading enzyme and at least one of a carrier and diluent wherein said composition is adapted to be administered to human and non-human on a periodic basis in less than a lethal dosage.

The claims relates to a composition for treating a disease comprising oxalate degrading enzyme and at least one of a carrier wherein said composition is adapted to be administered to human and non-human on a periodic basis in less than a lethal dosage, wherein the oxalate degrading enzyme is oxalic acid oxidase.

The claims relates to a composition for treating a disease comprising oxalate degrading enzyme and at least one of a carrier and diluent wherein said composition is adapted to be administered to human and non-human on a periodic basis in less than a lethal dosage, wherein the oxalate degrading enzyme is oxalic acid oxidase.

This invention also relates to a composition for treating a disease comprising oxalate degrading enzyme and at least one of a carrier and diluent. wherein said composition is adapted to be administered to human and non-human on a periodic basis in less than a lethal dosage, wherein the oxalate degrading enzyme is oxalate decarboxylase.

This invention also relates to a composition for treating a disease comprising oxalate degrading enzyme and at least one of a carrier wherein said composition is adapted to be administered to human and non-human on a periodic basis in less than a lethal dosage, wherein the oxalate degrading enzyme is oxalate decarboxylase.

This invention also relates to a composition for treating a disease comprising oxalate degrading enzyme and at least one of a carrier and diluent wherein said composition is adapted to be administered to human and non-human on a periodic basis in less than a lethal dosage, wherein the disease is selected from the group consisting of osteoporosis, kidney-urinary tract stone disease (urolithiasis), vulvodynia, oxalosis associated with end-stage renal disease, cardiac conductance disorders, Crohn's disease, and other enteric disease states.

This invention also relates to a composition for treating a disease comprising oxalate degrading enzyme and at least one of a carrier wherein said composition is adapted to be administered to human and non-human on a periodic basis in less than a lethal dosage, wherein the disease is selected from the group consisting of osteoporosis, kidney-urinary tract stone disease (urolithiasis), vulvodynia, oxalosis associated with end-stage renal disease, cardiac conductance disorders, Crohn's disease, and other enteric disease states.

This invention also relates to a composition for treating a disease comprising oxalate degrading enzyme and at least one of a carrier wherein said composition is adapted to be administered to human and non-human on a periodic basis in less than a lethal dosage, wherein the oxalate degrading enzyme is present in a group of natural foods, processed foods, beverages, liquids, and juices.

This invention also relates to a composition for treating a disease comprising oxalate degrading enzyme and at least one of a carrier and diluent wherein said composition is adapted to be administered to human and non-human on a periodic basis in less than a lethal dosage, wherein the oxalate degrading enzyme is present in a group of natural foods, processed foods, beverages, liquids, and juices.

This invention also relates to a composition for treating a disease comprising oxalate degrading enzyme and at least one of a carrier wherein said composition is adapted to be administered to human and non-human on a periodic basis in less than a lethal dosage, wherein the disease is selected from the group consisting of osteoporosis, kidney-urinary tract stone disease (urolithiasis), vulvodynia, oxalosis associated with end-stage renal disease, cardiac conductance disorders, Crohn's disease, and other enteric disease states.

This invention also relates to a composition for treating a disease comprising oxalate degrading enzyme and at least one of a carrier and diluent wherein said composition is adapted to be administered to human and non-human on a periodic basis in less than a lethal dosage, wherein the disease is selected from the group consisting of osteoporosis, kidney-urinary tract stone disease (urolithiasis), vulvodynia, oxalosis associated with end-stage renal disease, cardiac conductance disorders, Crohn's disease, and other enteric disease states.

This invention also relates to developing a method for addressing environmental concerns that are gaining importance in all fields, including the production of pulp and paper. In particular, the recent transition to oxygen containing bleaching agents together with the efforts to essentially close the processes—i. e. to minimize the effluents from the plants by recirculation of process liquids and reusing process chemicals—has given rise to new problems. One of these is the accumulation of organic and inorganic material. A particularly urgent problem is the formation of sparingly soluble oxalate compounds, which precipitate to form sediments and incrusts e. g. in digesters, evaporators, heat exchangers, pipe lines and washing filters, a phenomenon also known as scaling. Therefore, we have developed a product and a method for lowering of the concentration of oxalic acid in process liquids in the production of pulp and paper. The present invention concerns the lowering of the concentration of oxalic acid and in particular the prevention of the formation of calcium oxalate incrust and/or the degrading of precipitated calcium oxalate in the production of pulp and paper. These incrusts lead to lower heat transfer and change the flow characteristics, thus impairing the process economy. In some cases, parts of said incrusts can break off in chunks, which can travel down-stream and seriously damage valuable process equipment and cause costly shut-downs. The importance of reducing the level of oxalic acid has been addressed in a number of references such as WO9807922. The full content of WO9807922 is herein incorporated by the reference.

As indicated above, oxalic acid occurs abundantly in various plants, the most well known examples being rhubarb and spinach. Nevertheless it also occurs in significant amounts in wood, in concentrations in the range of about 0.1 to 0.4 kg/ton. The bark may contain up to 10-fold higher concentrations and hardwood bark can contain even up to about 15 kg oxalic acid per ton. This underlines the importance of using thoroughly debarked wood for the production of pulp. The main portion of the problem caused by oxalic acid is however related to its formation in the process, namely during pulping and bleaching. Examples of the underlying reactions were explained further in the description in WO9807922.

In the pulp and paper industry, this problem is currently attacked mainly in two ways, on one hand mechanical removal of the incrusts and, on the other hand various methods for changing the conditions governing the formation of incrusts. The former includes time consuming, often manual or semi-manual cleaning or scrubbing of the inside of equipment, e g pipes, screens and filters with water under high pressure. Sometimes this cleaning is supplemented with acid pretreatments or the use of complexing agents like EDTA. This approach is, under any circumstances, both cumbersome and costly. Costs are incurred both due to the added work required, the shut-down periods and increased wear on the equipment, e g screens. The latter approach, i. e. changing the conditions governing the formation of incrusts, includes the adjustment of pH, temperature and the addition of chemicals. Said chemicals either form a complex with one of the ions involved or inhibit the crystal-growth of calcium oxalate. The possibilities of manipulating the process parameters such as pH and temperature are restricted by process requirements and economical considerations. The addition of chemicals, among which in particular EDTA, DTPA, sodium tripolyphosphate, sodium pyrophosphate and methylene phosphoric acid are preferred, is followed by an increased COD (chemical oxygen demand), higher costs for chemicals and possible secondary process problems e g due to the accumulation of unwanted compounds. The development of trees with reduced amount of oxalate is one aspect of this invention offers a viable alternative to these methods. Transgenic trees with reduced oxalate is one way where the level of oxalate can be reduced where the transgenic trees express or overexpress oxalate-degrading enzymes. The use of transgenic plants that express or overexpress oxalate degarding enzyme(s) can be used to solve problems caused by the presence of oxalic acid in the brewing industry. U.S. Pat. No. 4,652,452 deals with this problem. According to U.S. Pat. No. 4,652,452, the level of oxalic acid must be lowered to less than about 15 ppm in finished beer to prevent “gushing”, whereby beer gushes from the bottle when opened. The precipitate of calcium oxalate, formed when calcium is added to stabilize and activate amylases during mashing, is also unwanted as the precipitating “beerstone” harbours contaminating microorganisms. U.S. Pat. No. 4,652,452 describes the introduction of oxalate decarboxylase (EC 4. 1.1.2) during mashing and/or fermentation to avoid the production of beerstone. However, U.S. Pat. No. 4,652,452 does not mention the use of transgenic plants that express or overexpress oxalate degrading enzymes in reducing the incidence of this problem in the brewing industry. In one aspect of this invention transgenic barley and other cereal plant species or their extract that express or overexpress oxalate-degrading enzyme is part of this invention and offers a viable alternatives in many aspects.

In that respect, this invention also relates to a method for lowering the level of oxalic acid and/or the amount of precipitated oxalate in the environment, wherein said method comprises the use of a plant, plant cell or any plant part. More preferably, the plant, plant cell or any plant part express or overexpress oxalate-degrading enzyme. The oxalate degrading enzyme can be derived from or present in, but not limited to, plant, bacteria, or fungi. More preferably, the plant, plant cell or any plant part is from cereal plant. More preferably, the cereal is a wheat, a barley, a maize, an oat, a sorghum, rice, a grass, a turfgrass, or a forage grass. In one aspect of the invention, the enzyme is produced recombinantly.

This invention also relates to a method for lowering the level of oxalic acid and/or the amount of precipitated oxalate in process liquids in the production of pulp and paper, wherein said method comprises the use of a plant, plant cell or any plant part. More preferably, the plant, plant cell or any plant part express or overexpress oxalate-degrading enzyme. The oxalate degrading enzyme can be derived from or present in, but not limited to, plant, bacteria, or fungi. More preferably, the plant, plant cell or any plant part is from cereal plant. More preferably, the cereal is a wheat, a barley, a maize, an oat, a sorghum, rice, a grass, a turfgrass, or a forage grass. In one aspect of the invention, the enzyme is produced recombinantly.

This invention also relates to a method for lowering the level of oxalic acid and/or the amount of precipitated oxalate in process liquids in the production of pulp and paper, in particular in preventing the forming of incrusts and sediments of oxalate compounds in the production of pulp and paper using oxidative bleaching, characterized in that the concentration of oxalic acid, present in the process liquid or liquids, is lowered to a value where no precipitation occurs at the given conditions by subjecting said process liquid or liquids to the action of at least one oxalic acid or oxalate degrading enzyme or a mixture of such enzymes wherein said method comprises the use of a plant, plant cell or any plant part. More preferably, the plant, plant cell or any plant part express or overexpress oxalate-degrading enzyme. The oxalate degrading enzyme can be derived from or present in, but not limited to, plant, bacteria, or fungi. More preferably, the plant, plant cell or any plant part is from cereal plant. More preferably, the cereal is a wheat, a barley, a maize, an oat, a sorghum, rice, a grass, a turfgrass, or a forage grass.

This invention also relates to a method for lowering the level of oxalic acid and/or the amount of precipitated oxalate in process liquids in the production of pulp and paper, characterized in that said oxalic acid or oxalate degrading enzyme is selected from the group consisting of formyl-CoA transferase, oxalyl-CoA decarboxylase, oxalic acid oxidase, oxalate decarboxylase and a mixture thereof.

This invention also relates to a method for lowering the level of oxalic acid and/or the amount of precipitated oxalate in process liquids in the production of pulp and paper, characterized in that said enzyme or enzymes is/are brought in contact with the process liquid or liquids by leading said process liquid or liquids through a vessel containing said enzyme or enzymes in an immobilized form wherein said method comprises the use of a plant, plant cell or any plant part.

This invention also relates to a method for lowering the level of oxalic acid and/or the amount of precipitated oxalate in process liquids in the production of pulp and paper, characterized in that said process liquid or liquids are subjected to at least one species of viable microorganisms expressing said enzyme or enzymes. More preferably, the microorganism is a bacterium of the group consisting of Clostridium, Pseudomonas, and oxalobacter. In one aspect of the invention, the enzymes are produced recombinantly.

This invention also relates to a method for lowering the level of oxalic acid and/or the amount of precipitated oxalate in process liquids in the production of pulp and paper, characterized in that said process liquid or liquids are subjected to biological material containing said enzyme or enzymes wherein the biological material is selected from the group consisting of a bacterium, a fungus, a plant or any other microorganism or any part thereof. These biological materials may be transgenic or non-transgenic materials.

This invention also relates to a method for lowering the level of oxalic acid and/or the amount of precipitated oxalate in process liquids in the production of pulp and paper, characterized in that the oxalic acid is converted to any one of formic acid, hydrogen peroxide, carbon dioxide and a mixture thereof.

Following are examples which illustrate procedures for practicing the invention. These examples should not be construed as limiting. This invention can be practiced with a number of plant species, but not limited to, corn, rapeseed, alfalfa, rice, rye, millet, pearl millet, proso, foxtail millet, finger millet, sunflower, safflower, wheat, soybean, tobacco, potato, peanuts, cotton, potato, cassava, coffee, coconut, pineapple, cocoa, banana, avocado, fig, guava, mango, olive, papaya, cashew, macadamia, almond, sugar beets, sugarcane, oats, duckweed, barley, vegetables, ornamentals, conifers, tomatoes, green beans, lima beans, peas, Citeltillis (including cucumber, cantaloupe, and musk melon), azalea, hydrangea, hibiscus, roses, tulips, daffodils, petunias, carnation, poinsettia, chrysanthemum, legumes (including peas and beans, such as guar, locust bean, fenugreek, soybean, garden beans, cowpea, mungbean, lima bean, fava bean, lentils, chickpea), peanuts, crown vetch, hairy vetch, adzuki bean, mung bean, and chickpea, lupine, trifolium, field bean, clover, Lotus, trefoil, lens, lentil, false indigo, alfalfa, orchard grass, tall fescue, perennial ryegrass, creeping bent grass, redtop grass, aneth, artichoke, blackberry, canola, cilantro, clementines, eucalyptus, fennel, grapefruit, honey dew, jicama, kiwifruit, lemon, lime, mushroom, nut, okra, orange, parsley, persimmon, plantain, pomegranate, poplar, radiata pine, Southern pine, sweetgum, tangerine, triticale, vine, yams, apple, pear, quince, cherry, apricot, melon, hemp, buckwheat, grape, raspberry, chenopodium, blueberry, nectarine, peach, plum, watermelon, eggplant, pepper, cauliflower, broccoli, onion, carrot, leek, beet, broad bean, celery, radish, pumpkin, endive, gourd, garlic, snapbean, squash, turnip, asparagas, and zuchini.

### EXAMPLE 1

transgenic plants with increased resistance to the destructive oxalic acid-producing pathogens such S. sclerotorium and then determining the mechanism of action for this resistance were established. A number of plants species, tomato, tobacco, arabidopsis, and cotton were transformed with oxalate oxidase. All of them were found to be resistant to toxic level of 200 mM of oxalate in vitro (leaf disk assay) and to S. sclerotorium when in tested at the plant (in vivo).

Oxalate (oxalic acid) is a diff-usable toxin associated with various plant diseases, particularly those caused by fungi. While some leafy green vegetables, including spinach and rhubarb, produce oxalate as a nutritional stress factor, certain pathogens synthesize and export large amounts of oxalate to assist in the establishment and spread of the organism throughout infected hosts. Oxalate is used by pathogens to gain access into and subsequently throughout an infected plant. See for example, Mehta and Datta, J Biol. Chem., 266: 23548-23553, and published PCT Application WO 92/14824 published in Sep. 3, 1992. Field crops such as sunflower, bean, canola, alfalfa, soybean, flax, safflower, peanut, clover, maize, sorghum, wheat, rice, as well as numerous vegetable crops, flowers, and trees are susceptible to oxalate-secreting pathogens. For example, fungal species including, but not limited to, Sclerotinia, Sclerotium, Aspergillus, Streptomyces, Penicillium, Pythium, Pacillus, Mycena, Leucostoma, Rhizoctonia and Schizophyllum use oxalic acid to provide an opportunistic route of entry into plants, causing serious damage to crops such as sunflower. Diseases of plants have caused an ongoing and constant problem in plant cultivation. The fungal pathogen, Sclerotinia sclerotiorum, in particular is said to cause disease in nearly 400 plant species. Sclerotinia sclerotiorum appears to be among the most nonspecific, omnivorous, and successful of plant pathogens. (Purdy, L. H., Phytopathology 69: 875-880 (1979)). Sclerotinia infections in sunflower, for example, are considered the major disease problems of the crop yet little genetic resistance is currently available to breeding programs to combat the various forms of this fungal infection. In fact, there are no major gene resistance mechanisms that have been defined in any species affected by this pathogen.

Enzymes that utilize oxalate as a substrate have been identified. These include oxalate oxidase (wheat oxalate oxidase is sometimes called germin) and oxalate decarboxylase. Oxalate oxidase catalyzes the conversion of oxalate to carbon dioxide and hydrogen peroxide. A gene encoding barley oxalate oxidase has been cloned from a barley root cDNA library and sequenced (See: PCT publication No. WO 92/14824, published in Sep. 3, 1992). A gene encoding wheat oxalate oxidase activity has been isolated and sequenced, and the gene has been introduced into a canola variety (PCT publication No. WO 92/15685 published in Sep. 17, 1992, Drawtewka-Kos, et al., J Biol. Chem., 264 (9): 4896-4900 (1991)). Oxalate decarboxylase converts oxalate to carbon dioxide and formic acid. A gene encoding oxalate decarboxylase has been isolated from Collybia velutipes (now termed Flammulina velutipes) and the cDNA clone has been sequenced (WO 94/12622, published in Jun. 9, 1994). In addition, another oxalate decarboxylase gene has been isolated from Aspergillus phoenices (U.S. patent application Ser. No. 08/821,827, now U.S. Pat. No. 6,297,425 filed on Mar. 21, 1997).

Construction of transgenic plants that express oxalate oxidase. An oxalate oxidase cDNA from wheat, that was kindly provided by Dr. Byron Lane, University of Toronto, was inserted into the_expression vector pRLT2 between the cauliflower mosaic virus (CaMV) enhanced 35S promoter and 35S terminator (Restrepo et al. 1990). The gene cassette was transferred to the binary plant transformation vector pCGN1578 (provided by Calgene) to create pCGN-oxalate oxidase. This plasmid was mobilized to Agrobacterium tumefaciens strain EH101 and used to produce transgenic tobacco plants (Nicotiana tabacum L. cv. Xanthi-NN) as described by Horsch et al. (1985). Northern blots performed using oxalate oxidase cDNA clone as a probe on RNA isolated from transgenic plants indicated that two independent transgenic plants produced significant amounts of oxalate oxidase mRNA and these plants had high levels of oxalate oxidase activity.

Transgenic plants that overexpress oxalate oxidase have been produced in tobacco, Arabidopsis, tomato, cotton. Plants that could be transformed and made disease resistant include, but in no way are limited to; sunflower, bean, canola, alfalfa, soybean, flax, safflower, peanut, clover, maize, sorghum, wheat, rice, as well as numerous vegetable crops, flowers, and trees. Other details of the transformation with gene that expresses oxalate-degrading enzymes and/or genes that causes the expression of enzymes that mediate the scavenge or the release of reactive oxygen species are explained in U.S. Pat. No. 6,403,861. There are a number of enzymes that are capable of producing hydrogen peroxide or a reactive oxygen species, for example but not limited to, glucose oxidase, choline oxidase, galactose oxidase, L-aspartate oxidase, xanthine oxidase, monoamine oxidase, eosinophil peroxidase, glycolate oxidase, polyamine oxidase, copper amine oxidase, flavin amine oxidase, berberine Bridge Enzyme, choline oxidase, acyl coA oxidase, amino cyclopropane carboxylate oxidase (ACC oxidase), pyridoxamine-phosphate oxidase, sarcosine oxidase, sulfite oxidase, methyl sterol oxidase, aldehyde oxidase, xanthine oxidase, NADPH oxidase (respiratory burst enzyme homolog), large subunit (GP91) and most preferably, oxalate oxidase. The complete content of U.S. Pat. No. 6,403,861 is incorporated by a reference.

In one part of the invention, transgenic plants comprising gene(s) that expresses enzymes that mediate the scavenge or the release of reactive oxygen species with or without oxalate degrading enzymes are also part of the invention.

Oxalic acid tolerance assays. Leaf disks from oxalate oxidase-expressing transgenic plants and control plants were incubated in 200 mM oxalic acid solution (pH 4.0) for 48 hrs. In this solution, leaf disks from control plants turn completely brown in 2 to 3 days. The degree of damage was assessed by visually and by measuring chlorophyll degradation. There was a substantial loss of chlorophyll in extracts from leafs disks of control plants relative to the oxalate oxidase-expressing plants. Sclerotinia infection assays. Sclerotinia sclerotiorum, cause of damping off and stem diseases (Agrios, 1986). The fungus was maintained on potato-dextrose-agar (PDA) medium for mycelium growth and sclerotia formation. Leaf disks from oxalate oxidase-expressing plants and control plants were inoculated on plates that contained a PDA plug with mycelia. Three days after incubation with the fungus, the plates were entirely covered with mycelia. Visual assessment of the leaf disks after 3 days showed complete infection and loss of chlorophyll and turgor in leaf disks from the control plant; whereas, disks from the high oxalate oxidase expressing plant line 2D showed little damage.

Development of transgenic plants that express antioxidative enzymes. A number of transgenic plant lines that express a variety of enzymes that scavenge reactive oxygen intermediates or are involved in the regeneration of low molecular weight antioxidant molecules have been developed and tested. Transgenic tobacco lines that express the following transgenes have been developed and tested: 1) various isoforms of SOD that are targeted to different cellular compartments, 2) cytosolic and chloroplastic forms of APX, 3) peroxisomal and apoplastic forms of catalase, 4) glutathione-S transferase, 5) a putative glutathione peroxidase, and 6) glutathione reductase. Homozygous expressing plant lines have been developed in most cases.

To remove hydrogen peroxide, the by-product of decomposing oxalic acid by oxalic acid oxidase, transgenic plants that overexpress oxalic acid oxidase can be further transformed with nucleic acid sequence which confers upon expression the production of hydrogen peroxide scavenging enzymes that lead to alleviation or the scavenging of hydrogen peroxide. These transgenic plants can be produced as mentioned above. In one aspect of the invention, genes expressing enzymes that degrade oxalate and hydrogen peroxide can be co-transformed into a host cell.

The hydrogen peroxide can also be disassembled by boiling or by adding catalase (for example, but not limited to) directly to the food products. In one aspect of this invention, the hydrogen peroxide scavenging enzymes can be added through commercially available products or through the ingestion of food that contains these hydrogen peroxide scavenging enzymes.

Transfer by Plant Breeding

Once a single transformed plant has been obtained by the foregoing recombinant DNA method, conventional plant breeding methods can be used to transfer the gene and associated regulatory sequences via crossing and backcrossing. Such intermediate methods will comprise the further steps of: (1) sexually crossing the oxalate-degrading enzyme expressing or overexpressing plant with a plant from the oxalate-degrading low expressing or not expressing transgenic taxon; (2) recovering reproductive material from the progeny of the cross; and (3) growing oxalate-degarding enzyme expressing or overexpressing plants from the reproductive material. Where desirable or necessary, the agronomic characteristics of the susceptible taxon can be substantially preserved by expanding this method to include the further steps of repetitively: (1) backcrossing the the oxalate-degrading enzyme expressing or overexpressing progeny with the oxalate-degrading enzyme not expressing or underexpressing plant from taxon; and (2) selecting for expression of a hydrogen peroxide producing enzyme activity (or an associated marker gene) among the progeny of the backcross, until the desired percentage of the characteristics of the susceptible taxon are present in the progeny along with the gene or genes imparting oxalic acid degrading and/or hydrogen peroxide enzyme activity. By the term “taxon” herein is meant a unit of botanical classification. It thus includes, genus, species, cultivars, varieties, variants and other minor taxonomic groups which lack a consistent nomenclature.

1. A method for treating, preventing, controlling, or impeding
a disease in a human and non human wherein said method comprises administering to said human a composition comprising a material selected from the group consisting of oxalate-degrading microbes and oxalate-degrading enzymes.

2. The method, according to claim 1, wherein said method comprises administration of oxalate-degrading enzymes, wherein the oxalate degrading enzyme is selected from the group consisting of formyl-CoA transferase, oxalyl-CoA decarboxylase, oxalic acid oxidase and oxalate decarboxylase.

3. The method, according to claim 1, wherein said oxalate-degrading enzymes are obtained from bacteria, fungi or plant.

4. A composition for treating, preventing, controlling, or impeding
a disease in a human and non human wherein said composition comprises a material selected from the group consisting of oxalate-degrading microbes and oxalate-degrading enzymes formulated in a food product for human oral consumption.

5. The composition, according to claim 4, wherein said composition comprises whole, viable oxalate-degrading bacteria.

6. The method, according to claim 4, wherein said composition comprises cell lysate of oxalate-degrading bacteria.

7. The composition, according to claim 4, wherein said bacteria are Oxalobacter formigenes.

8. The composition, according to claim 4, wherein said bacteria are selected from the group consisting of Clostridium and Pseudomonas.

9. The composition, according to claim 4, wherein said composition comprises oxalate-degrading enzymes.

10. The composition, according to claim 9, wherein said enzymes are formyl-CoA transferase and oxalyl CoA decarboxylase, oxalic acid oxidase or oxalate decarboxylase.

11. The composition, according to claim 10, which further comprises a compound selected from the group consisting of oxalyl CoA, MgCl₂, and TPP.

12. The composition, according to claim 4, wherein said composition is formulated to reduce deactivation in the stomach.

13. The composition, according to claim 12, wherein said composition is coated with a material which preferentially degrades in the small intestine.

14. The method, according to claim 1, wherein said method is used to treat osteoporosis.

15. The method, according to claim 1, wherein said method is used to treat osteoporosis.

16. The method, according to claim 1, wherein the oxalate concentration is reduced in the intestines, blood, or urine.

17. A method for treating, preventing, controlling, or impeding
a disease in a human and non human wherein said method comprises administering to said human a composition comprising a material selected from the group consisting of oxalate-degrading microbes and oxalate-degrading enzymes; wherein the disease is selected from the group consisting of osteoporosis, urolithiasis, vulvodynia, and oxalosis and alike diseases.

18. The method, according to claim 17, wherein the oxalate-degrading enzyme is selected from the group consisting of a bacterium, a fungus, or a plant.

19. The method, according to claim 17, wherein said method comprises administration of oxalate-degrading enzymes wherein the oxalate de-grading enzymes is selected from the group consisting of formyl-CoA transferase, oxalyl-CoA decarboxylase, oxalic acid oxidase and oxalate decarboxylase.

20. The method, according to claim 3, wherein said oxalate-degrading enzymes are derived from bacteria of the group consisting of Clostridium, Pseudomonas, and oxalobacter.
